(19) United States
(12) Patent Application Publication
(10) Pub. No.: US 2024/0374867 A1
(43) Pub. Date: Nov. 14, 2024

(54) STEERABLE CATHETER SYSTEM

(71) Applicant: Endoron Medical Ltd., Kfar Saba (IL)

(72) Inventor: Eyal TEICHMAN, Hod HaSharon (IL)

(73) Assignee: Endoron Medical Ltd., Kfar Saba (IL)

(21) Appl. No.: 18/687,907

(22) PCT Filed: Oct. 4, 2022
(86) PCT No.: PCT/IL2022/051058

§ 371 Date: Feb. 29, 2024

Related U.S. Application Data
(60) Provisional application No. 63/252,648, filed on Oct. 6, 2021.

(51) Int. Cl. A61M 25/01

(52) U.S. Cl. CPC A61M 25/0147; A61M 25/0136

(57) ABSTRACT

A system for positioning an intraluminal device includes a catheter having an elongated catheter body having a distal portion configured for delivering the intraluminal device and a catheter tube having a wire spanning a length thereof. The system also includes a handle attached to the elongated catheter body and having at least one steering mechanism configured for pushing the catheter tube in a distal direction thereby steering the distal portion of the elongated catheter body and positioning the intraluminal device for delivery.

### RELATED APPLICATION

This application claims the benefit of priority of U.S. Provisional Patent Application No. 63/252,648 filed on 6 Oct. 2021, the contents of which are incorporated herein by reference in their entirety.

### BACKGROUND

The present invention relates to steerable catheter system that can be used to position and deliver an intraluminal device. Embodiments of the present invention relate to a steerable catheter system for positioning and delivering a stent graft or a stent graft fixation device at a site of an aortic aneurysm.

Vascular aneurysms are characterized by abnormal dilation of a blood vessel that typically results from weakening of the arterial wall caused by disease or genetic predisposition. Aneurysms have been commonly treated by open surgical procedures in which the diseased vessel segment is bypassed or covered with a protective graft.

In more recent years, open procedures have been replaced with minimally invasive procedures that utilize endoluminal stent grafts that enable blood flow through the vessel while bypassing the aneurysm site.

Such stent grafts typically include a metallic support structure carrying a graft material such as Dacron, or polytetrafluoroethylene (PTFE). The graft material is sealed against the vessel wall by the support structure.

Although effective in sealing off the aneurysm, stent grafts can migrate over time due to the force associated with the blood flowing through the stent graft and the expansion and contraction of the arteries due to the pulsation of blood therethrough. Such migration can lead to leakage of blood into the aneurysm site.

Anchors for tissue fixation and stents carrying such anchors have been developed in order to prevent stent graft migration. PCT publication WO2019239409 to the present inventors describes a graft securing system for preventing stent graft migration.

While stent grafts and securing/fixation solutions can be delivered to the site of treatment using percutaneous approaches, correctly positioning such intraluminal devices at the site of deployment can be challenging due to vessel curvature near or at the site of deployment.

There is thus a need for, and it would be highly advantageous to have, a catheter system that can be used to accurately position an intraluminal device at the site of delivery.

### SUMMARY

According to one aspect of the present invention there is provided a system for positioning an intraluminal device comprising: a catheter having an elongated catheter body, the elongated catheter body having a distal portion configured for delivering the intraluminal device and including a catheter tube having a wire spanning a length thereof; and a handle attached to the elongated catheter body and having at least one steering mechanism configured for pushing the catheter tube in a distal direction thereby steering the distal portion of the elongated catheter body and positioning the intraluminal device for delivery.

According to embodiments of the present invention the at least one steering mechanism is also configured for pulling the wire in a proximal direction.

According to embodiments of the present invention a first end of the wire is attached to a distal end of the catheter tube and further wherein the second end of the wire is attached to the at least one steering mechanism.

According to embodiments of the present invention the at least one steering mechanism includes a first translatable element attached to the second end of the wire and a second translatable element attached to a proximal portion of the catheter tube.

According to embodiments of the present invention at least one steering mechanism further includes a rotatable knob for translating the first and the second translatable elements in opposite directions.

According to embodiments of the present invention the system further comprises a sheath covering a distal portion of the elongated catheter body.

According to embodiments of the present invention the handle includes a sheath retraction mechanism.

According to embodiments of the present invention the system further comprises a nose cone at a distal end of elongated catheter body.

According to embodiments of the present invention the handle includes a nose cone extension mechanism.

According to embodiments of the present invention the system further comprises the intraluminal device, wherein a proximal portion of the intraluminal device is covered by the sheath and a distal portion of the intraluminal device is covered by the nose cone.

According to one aspect of the present invention there is provided a method of positioning an intraluminal device in a lumen of a vessel comprising: advancing into the lumen of a vessel a catheter having an elongated catheter body having a distal portion carrying the intraluminal device, the elongated catheter body including a catheter tube having a wire spanning a length thereof; and steering the distal portion of elongated catheter body via at least one steering mechanism configured for pushing the catheter tube in a distal direction thereby positioning the intraluminal device for delivery.

According to embodiments of the present invention, steering is also carried out by pulling the wire in a proximal direction.

According to embodiments of the present invention a first end of the wire is attached to a distal end of the catheter tube and further wherein the second end of the wire is attached to the at least one steering mechanism.

According to embodiments of the present invention the at least one steering mechanism includes a first translatable element attached to the second end of the wire and a second translatable element attached to a proximal portion of the catheter tube.

According to embodiments of the present invention at least one steering mechanism further includes a rotatable knob for translating the first and the second translatable elements in opposite directions.

According to embodiments of the present invention a proximal portion of the intraluminal device is covered by a sheath and a distal portion of the intraluminal device is covered by a nose cone.

According to embodiments of the present invention the method further comprises advancing the nose cone and retracting the sheath to thereby expose the intraluminal device for the delivery.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B illustrate the steerable catheter system of the present invention with the elongated catheter body in a straight orientation (FIG. 1A) and a deflected (steered) orientation (FIG. 1B).

FIG. 2 illustrates the handle steering mechanism of the present catheter system.

FIGS. 3A-E illustrate deployment of an intraluminal device from the present catheter system.

FIGS. 4A-B illustrate an intraluminal device in the collapsed (in-catheter) state (FIG. 4A) and the deployed (in-vessel) state (FIG. 4B).

### DETAILED DESCRIPTION

The present invention is of a steerable catheter system that can be used to position and deploy an intraluminal device at the site of treatment. Specifically, the present invention can be used to position and deploy a graft securing device within an aorta.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Correctly positioning stents, stent grafts and stent graft fixation devices can be challenging due to vessel curvature near or at the site of deployment. For example, a femoral percutaneous approach to an abdominal aortic aneurysm (AAA) site is curved along two axis and thus can severely limit the ability of the physician to symmetrically position the stent graft or the graft fixation device along opposing vessel walls.

While reducing the present invention to practice, the present inventors have devised a steerable catheter system that can be used to correctly position and deploy an intraluminal device in torturous vessel anatomy.

Thus, according to one aspect of the present invention there is provided a system for positioning an intraluminal device.

As used herein, the phrase “intraluminal device” refers to any device positionable within a lumen of, for example, a blood vessel, a ureter, a urethra, a duct, a GI tract, an organ and the like.

The system includes a catheter having an elongated catheter body having a distal portion configured for delivering the intraluminal device. The elongated catheter body includes a catheter tube having a wire spanning a length thereof.

The system further includes a handle attached to the elongated catheter body, the handle includes at least one steering mechanism configured for pushing the catheter tube in a distal direction to thereby deflect (steer) the distal portion of the elongated catheter body and position the intraluminal device for delivery. The steering mechanism can also be configured for pulling the wire (proximally) when the catheter tube is pushed (distally).

While experimenting with several steerable catheter designs (see examples section) the present inventors uncovered that a wire-steerable catheter tube shortens when deflected due to the compressive forces applied by the wire when pulled. For example, a braided re-enforced catheter tube made of Pebax and being about 700 mm in length, 4.5 mm diameter and having a 0.6 mm wall thickness would shorten 7-8 mm at the distal end when steered via wire pull.

Since such shortening can lead to unintentional device deployment and/or device mispositioning, the present inventors sought out solutions that would enable steering without these aforementioned limitations.

The present inventors discovered that pushing the steerable tube (catheter tube) from the proximal end (at handle) results in compression at the proximal end of the tube and negligible forward movement (elongation) at the distal end of the tube while simultaneous wire pull and catheter tube push resulted in no appreciable movement at the distal end of the catheter tube.

When the same catheter tube (described above) was steered via proximal advancement (push) of the catheter tube, the tube moved in a distal direction about 4 mm while simultaneous tube push and wire pull resulted in no movement at the distal end.

Thus, the at least one steering mechanism can be configured for pushing the catheter tube in a distal direction to thereby deflect (steer) the distal portion of the elongated catheter body and position the intraluminal device for delivery. The steering mechanism can also be configured for pulling the wire (proximally) when the catheter tube is pushed (distally).

The steering mechanism can include one or more knobs for counter-rotating two translatable elements (simultaneously or separately), a first translatable element for pushing the catheter tube and a second translatable element for pulling the wire attached to the distal end of the catheter tube.

According to one embodiment of the present invention, the steering mechanism includes a single knob for simultaneously translating both (first and second) translatable elements in opposite directions.

According to another embodiment of the present invention, the steering mechanism includes two knobs for separately translating the first and second translatable elements in opposite directions. Such a configuration can be advantageous under conditions wherein changes to the distal end length can be used for micro adjustments to the position of the delivered intraluminal device. While use of a single steering mechanism to control both catheter tube advancement and wire pull is preferred, a configuration of the present system having two separate steering mechanisms one for controlling catheter tube advancement and the other for pulling the wire is also envisaged herein.

A more detailed description of one embodiment of the steering mechanism of the present invention is provided hereinbelow with reference to FIG. 2.

As is mentioned hereinabove, the present system can be used to position and delivery (deploy) and intraluminal device such as a graft fixation device.

To that end, the distal end portion of the elongated catheter body can be adapted to carry such a device and deploy it at a target location.

Depending on the device deployed, the present system can optionally include a sheath covering a distal portion of the elongated catheter body (for covering a portion of the device carried upon) and corresponding sheath retraction mechanism; and a nose cone at a distal end of the elongated catheter body and a corresponding nose cone extension mechanism. An additional bypass sheath for enabling controlled advancement through the introducer while minimizing device positioning errors during device unsheathing as result of delivery system friction with the introducer can also be provided.

In the configuration having the sheath and nose cone, the proximal portion of the intraluminal device can be covered by the sheath and the distal portion of the intraluminal device can be covered by the nose cone.

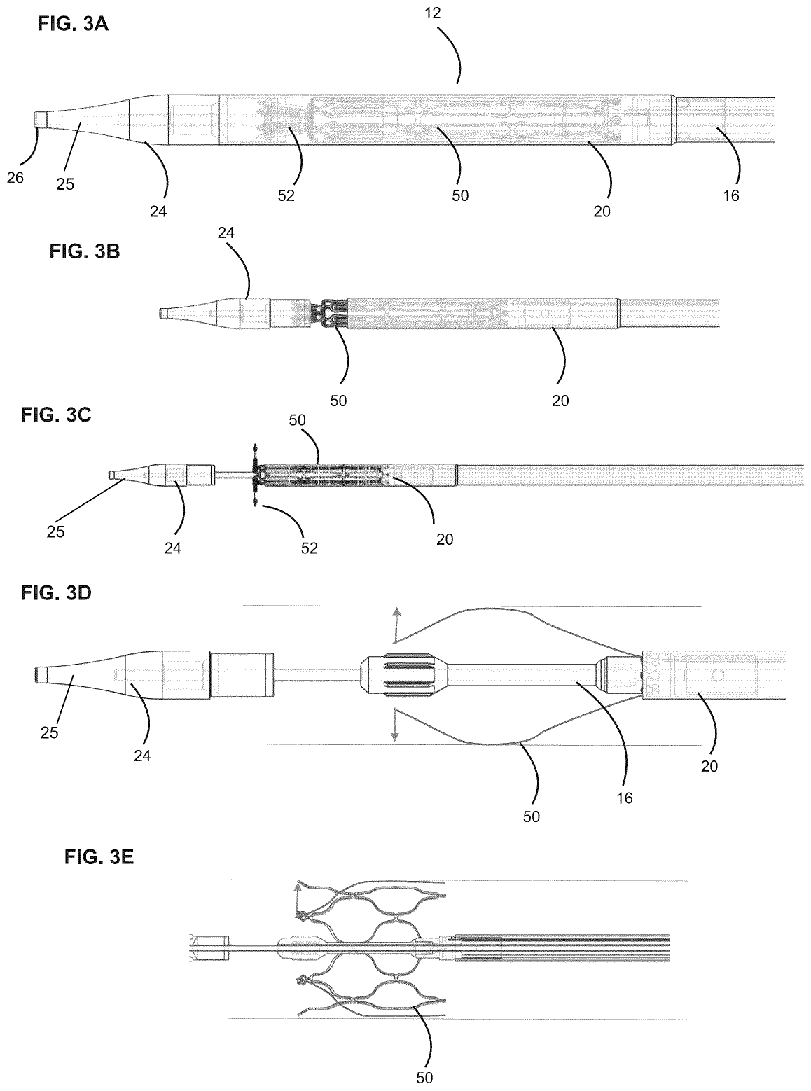

Device deployment from such a configuration of the present system is described below with reference to FIGS. 3A-E.

The present catheter system can further include ports for guide wire insertion and additional 2 flushing ports to enable lumen flushing with saline prior to device deployment.

The present catheter system can be used to position and deploy any intraluminal device in any lumen in the body of a subject.

One such device is a graft fixation device deployable within a stent-graft.

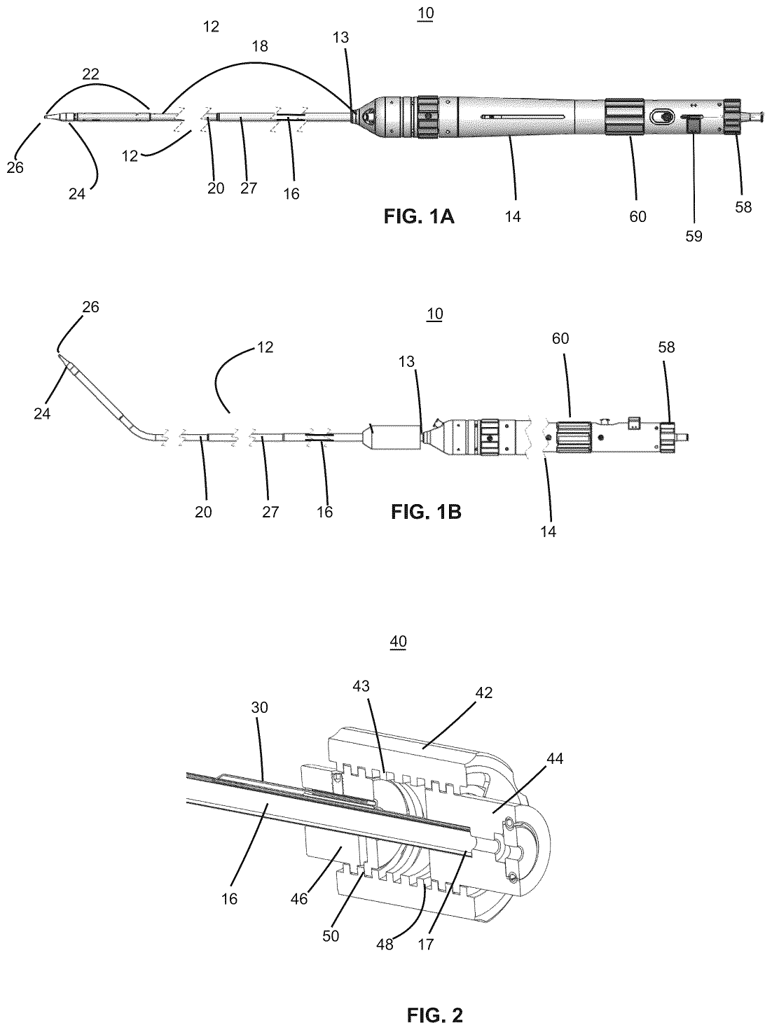

Referring now to the drawings, FIGS. 1A-B illustrate one embodiment of the present system which is referred to herein as system 10. FIGS. 1A-B illustrate the elongated catheter body of system in linear and deflected (steered) orientations (respectively).

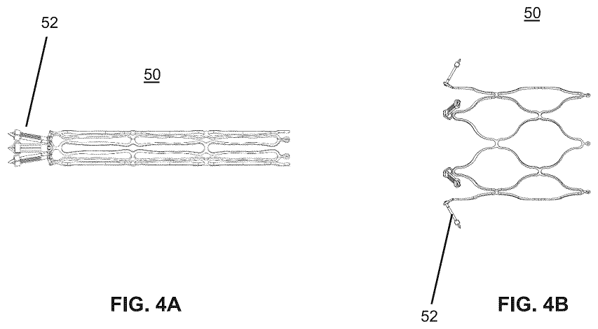

System 10 is configured for positioning and deploying a fixation device (FIG. 4A-B) within a stent graft used for endovascular aneurysm repair (EVAR). The fixation device includes a stent-like frame supporting radially deployable anchors designed for fixating the graft to aortic tissue. Embodiments of this device are described in detail in PCT publication WO2019239409.

System 10 includes an elongated catheter body 12 attached at proximal end 13 to a handle 14. Elongated catheter body 12 includes a catheter tube 16 that is covered at mid-proximal portion 18 by sheath 20 and at distal portion 22 by a nose cone 24.

Catheter body 12 includes a wire lumen that extends from handle 14 through tip 26 of nose cone 24, a steering lumen 16, an outer sheath 20 and a bypass sheath 27. Outer sheath 20 extends from handle 14 to the tip 26 and is 16 French (fr) in diameter at the proximal end and 18 fr at the distal end. Steering lumen 16 is connected (at distal end) to the portion of catheter body on which the device is mounted.

An overlap can be provided between the proximal end of nose cone 24 and the distal end of sheath 20. Bypass sheath 27 covers the proximal portion of sheath 20 and provides a shield” for minimizing interaction between sheath 20 and an introducer catheter during device positioning, rotation and unsheathing.

Tip 26 of nose cone 24 is configured for facilitating navigation of system 10 through the vasculature and for minimizing trauma to vascular tissue. Nose cone 24 can be connected via the wire lumen to the most proximal port and is translatable by knob 58. That catheter also provides a 0.035″ lumen for a guide wire.

The dimensions of the various components of system 10 can be as follows. Bypass sheath 27 can have an outer diameter (OD) of 6 mm (18 Fr), a wall thickness of 0.3 mm, a length of about 300 mm and can be made from braided Pebax. Sheath 20 can have an OD of 6 mm (18 fr) at the distal end and 5.3 mm (16 fr) at the proximal end, a wall thickness of 0.3 mm at the distal end and 0.3 mm at the proximal end, a distal portion length of 60 mm and a proximal portion length of 600 mm and can be made from braided Pebax.

Steering lumen 16 can be a tube fabricated from braided Pebax or Vestamid with an outer diameter of 4.5 mm, a wall thickness of 0.6 mm and a length of about 600 mm. The steering center of curvature can be 90 mm from tip 26. Nose cone 24 can be fabricated from stainless steel with tip 26 fabricated from low shore Pebax or nylon.

System 10 further includes at least one wire 30 (shown in FIG. 2) that is attached at its distal end to a distal end of catheter tube 16. Wire can run within catheter tube 16 or within a dedicated lumen in a wall of catheter tube 16. Catheter tube 16 can be 500-800 mm in length with an outer diameter of 4-4.5 mm and an inner (lumen) diameter of 1.5-3.3 mm. A proximal portion of catheter tube 16 (400-700 mm) can be stiffer than a distal portion thereof. The wall of catheter tube 16 can include a lumen for accommodating wire 30.

Wire 30 can be made from stainless steel about 0.3-0.4 mm in diameter, the lumen of wire 30 can be slightly oversized (2-5% larger in diameter). Although a single wire 30 can be used for steering catheter tube 16 (and as a result elongated catheter body 12), system 10 can include 2, 3, 4 or more wires each positioned through a dedicated lumen in a wall of catheter tube 16 and each separately operable from handle 14. A single wire 30 is advantageous in that it can be used (along with device rotation) to position tip 26 at any point within a radial plane with minimal system 10 complexity.

System 10 further includes a steering mechanism 40 in handle 14 for steering catheter tube 16 and hence catheter body 12.

In order to steer catheter body 12 steering mechanism 40 provides two functions, wire 30 pull and catheter tube 16 push. To that end, steering mechanism is attached to wire 30 and to proximal end 17 of catheter tube 16.

One configuration of steering mechanism 40 is shown in FIG. 2. Steering mechanism 40 includes a rotatable knob 42 which includes threads 43 for engaging two translatable elements 44 and 46 through threads 48 and 50 (respectively). Threads 48 and 50 are counter-spiraled to provide counter translation of elements 44 and 46 that are radially locked (i.e., they cannot rotate only mover longitudinally). Thus, when knob 42 is rotated clockwise (looking at knob from the proximal end of system 10), translatable element 44 translates (slides) forward (in a distal direction) and translatable element 46 translates (slides) back (in a proximal direction). Counterclockwise rotation of rotatable knob 42 can be used to linearize a deflected elongated catheter body 16. Tube 16 is not attached to element 46 and thus is capable of sliding thereagainst.

When translatable element 44 slides forward it pushes against proximal end 17 of catheter tube 16 applying a compressive force thereto. Such a force tensions wire 30 (anchored to translatable element 46) and deflects catheter tube 16 away from the side of catheter tube 16 (at which wire 30 is positioned).

Simultaneous backward translation of translatable element 46 pulls wire 30 and distal end of catheter tube 16 to which it is attached thereby enhancing deflection of catheter tube 16 and countering any distal movement of the distal end of catheter tube 16 caused by forward translation of translatable element 44. Thus, this push pull mechanism maintains the distal end of the steerable shaft in position with reference to the system handle and enables correct positional deployment of a device carried by the catheter.

While such a simultaneous push pull mechanism is preferred for the reasons stated above, a steering mechanism 40 capable of separate catheter tube 16 push and wire 30 pull (using, for example, two rotatable knobs) or a steering mechanism capable of only catheter tube 16 push can also be used in system 10.

System 10 can optionally include an indicator for quantifying the steering angle of the shaft. Such an indicator can include a “scale” for the rotatable knob (single time calibration of knob rotation to tip angle) or utilize a transparent material for the knob and a viewing window in the handle body such that the linear internal moving parts are viewable to the user and enable calibration of the linear gap change with distal end deflection.

The degree of deflection of elongated catheter body 12 can be controlled by the amount of rotatable knob 42 rotation and can be corrected by counter-rotation of rotatable knob 42. Rotatable knob 42 can include markings denoting the degree (angle) of deflection corresponding to knob 42 position. A lock button can be used to lock rotatable knob 42 at any position to allow rotation of system 10 without fear of a change in deflection.

Steering mechanism can alternatively include a lever-based translation mechanism in which the operator activates a lever that is connected to the sliders by set of connecting rods. In such a configuration, puling the lever in one direction will result a counter direction linear movement of both sliders and pushing it will reverse direction.

As is mentioned hereinabove, the configuration of system 10 shown in FIGS. 1A-B can be used to deliver a graft anchoring device 50 (FIGS. 4A-B). Such a device is preferably deployed in a stepwise manner in order to first expose distally-positioned radially deployable anchors 52 and then the rest of the device.

In order to enable such deployment, system 10 includes a nose cone 24 covering a distal portion of device 50 and a sheath 20 covering a proximal portion of device 50.

Forward deployment of nose cone 24 (to expose the distal portion of device 50 and deploy anchors 52) can be carried out by rotating a knob 58 of a handle mechanism. Knob 58 has an internal diameter (ID) thread that rides over an external threaded screw which is connected to the tip via a wire/rod/tube 25 (positioned through a lumen in catheter tube 16). The threaded portion is radially locked so once the knob is rotated the threaded part moves distally to wire/rod/tube 25 to translate tip 26 distally.

Pulling sheath 56 back (to expose and deploy the rest of device 50) can be carried out via a knob 60 of a sheath deployment mechanism. Knob 60 is constructed similarly to knob 58 but is used to pull rather than push a wire/rod in order to deploy sheath 56.

System 10 can also include a safety latch 59 for preventing any movement of nose cone 24. Safety latch 59 is removed once system 10 is correctly positioned in the body. Safety latch 59 is used to prevent any premature unintentional nose cone deployment that might cause premature deployment of anchors 52.

System 10 can also include one or more Luer ports for flushing (de-airing) of the bypass sheath and for flushing of the lumens of catheter tube 20 and elongated catheter body 12 as well as flushing of the lumen of the guide wire.

FIGS. 3A-E illustrate use of system 10 in deploying device 50 in a blood vessel such as a descending aorta. Deployment of device 50 can be carried out from a deflected or a linear catheter body 12. For the sake of simplicity, a linear orientation is shown in FIGS. 3A-E.

Following positioning of device 50 at a target location (FIG. 3A) using standard over-the-wire techniques, nose cone 24 is pushed forward (using knob 58) to deploy anchors 52 (FIGS. 3B-C). Once symmetric orientation of anchors 52 with respect to opposing vessel walls is confirmed (using fluoroscopy), sheath 20 is pulled back using knob 60 (FIGS. 3D-E). Once device 50 is completely deployed in the vessel, system 10 is collapsed and removed and a balloon catheter is inserted through device 50 and used to apply a radially outward force on device 50 thereby pushing anchors 52 through graft and tissue.

Deployment of device 50 in an aortic arch or the neck of an aneurism (AAA) requires steering (deflection) of the distal portion of system 10 due to the curved nature of these lumens and the need to center the deployed device prior to deployment.

If positioned using a non-steerable linear delivery catheter, the device will be skewed and will lay against one wall of the lumen thereby making it impossible to symmetrically position the device for deployment. As a result, the device will not align with the longitudinal axis of the lumen with one side higher than the other side (skewing).

In order to compensate for such skewed positioning, the delivery end of the catheter must be steered (deflected) in order to center the device within the lumen of the aorta. Once steering achieves correct positioning, the device can be safely deployed.

As used herein the term “about” refers to ±10%.

Additional objects, advantages, and novel features of the present invention will become apparent to one ordinarily skilled in the art upon examination of the following examples, which are not intended to be limiting.

### EXAMPLES

Reference is now made to the following examples, which together with the above descriptions, illustrate the invention in a non-limiting fashion.

### Example 1

### Steerable Catheter Design

A steerable catheter was tested by the present inventors in order to determine the extent of catheter tube shortening when the steerable portion of the catheter tube is deflected and devise a solution that would enable steering without unintentional device deployment and/or device mispositioning.

The steerable catheter included a tube fabricated from braided PEBAX with an internal diameter of 3.2 mm and an external diameter of 4.3 mm (wall thickness of 0.55 mm). A wire positioned through a dedicated lumen in the wall of the catheter tube was used to deflect the catheter tube and the extent of tube shortening was measured as a function of the pull force exerted (about 40 N) and the extent of deflection (over 60 degrees).

The pull wire proximal end travel was measured with reference to the proximal end of the catheter tube (12.7 mm) and the pull wire force (about 40 N) was measured for a full steering angle (over 60 degrees).

Following several rounds of measurements, the present inventors then determined that catheter tube shortening (5-6 mm) is linear with the proximal end travel of the pull wire and can be represented by the following formula:

$$L = F ( K 1 + K 2 ) / K 1 \times K 2$$

$$\begin{matrix}L = Pull wire backwards travel distance \\ F = the force required for full angle steering \\ K 1 = Pull wire spring coefficient ( elongation ) \\ K 2 = Steering shaft spring coefficient ( shortening )\end{matrix}$$

In order to compensate for such shortening the present inventors designed the present catheter system to simultaneously push the proximal end of the catheter shaft forward (in a distal direction) the same distance traveled by the pull the wire in a proximal direction (backwards) and both were designed to move half of the above measured distance (L/2).

### Example 2

### Testing of a Prototype System

A system as described in FIGS. 1A-B equipped with the steering mechanism described in FIG. 2 was constructed and tested for device displacement.

In general, the system included three coaxial lumens, a wire lumen, a steerable lumen and an outer sheath.

The test was carried out by measuring the distance between a predefined point on the steerable tube distal end and a predefined point on the outer sheath distal section while rotating the steering knob and deflecting the system distal end through full angle range (about 60 degrees).

Negligible relative movement (less than 0.5 mm) between the steerable shaft distal end and the outer sheath distal end was observed for the prototype system.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated hercin by reference in its/their entirety.

What is claimed is:

1. A system for positioning an intraluminal device comprising:
(a) a catheter having an elongated catheter body, said elongated catheter body having a distal portion configured for delivering the intraluminal device and including a catheter tube having a wire spanning a length thereof; and
(b) a handle attached to said elongated catheter body and having at least one steering mechanism configured for pushing said catheter tube in a distal direction;
thereby steering said distal portion of said elongated catheter body and positioning the intraluminal device for delivery.

2. The system of claim 1, wherein said at least one steering mechanism is also configured for pulling said wire in a proximal direction.

3. The system of claim 2, wherein a first end of said wire is attached to a distal end of said catheter tube and further wherein said second end of said wire is attached to said at least one steering mechanism.

4. The system of claim 3, wherein said at least one steering mechanism includes a first translatable element attached to said second end of said wire and a second translatable element attached to a proximal portion of said catheter tube.

5. The system of claim 4, wherein at least one steering mechanism further includes a rotatable knob for translating said first and said second translatable elements in opposite directions.

6. The system of claim 1, further comprising a sheath covering a distal portion of said elongated catheter body.

7. The system of claim 6, wherein said handle includes a sheath retraction mechanism.

8. The system of claim 7, further comprising a nose cone at a distal end of elongated catheter body.

9. The system of claim 8, wherein said handle includes a nose cone extension mechanism.

10. The system of claim 9, further comprising the intraluminal device, wherein a proximal portion of the intraluminal device is covered by said sheath and a distal portion of the intraluminal device is covered by said nose cone.

11. A method of positioning an intraluminal device in a lumen of a vessel comprising:
(a) advancing into the lumen of a vessel a catheter having an elongated catheter body having a distal portion carrying the intraluminal device, said elongated catheter body including a catheter tube having a wire spanning a length thereof; and
(b) steering said distal portion of elongated catheter body via at least one steering mechanism configured for pushing said catheter tube in a distal direction;
thereby positioning the intraluminal device for delivery.

12. The method of claim 11, wherein (b) is also carried out by pulling said wire in a proximal direction.

13. The method of claim 12, wherein a first end of said wire is attached to a distal end of said catheter tube and further wherein said second end of said wire is attached to said at least one steering mechanism.

14. The method of claim 13, wherein said at least one steering mechanism includes a first translatable element attached to said second end of said wire and a second translatable element attached to a proximal portion of said catheter tube.

15. The method of claim 14, wherein at least one steering mechanism further includes a rotatable knob for translating said first and said second translatable elements in opposite directions.

16. The method of claim 11, wherein a proximal portion of the intraluminal device is covered by a sheath and a distal portion of the intraluminal device is covered by a nose cone.

17. The method of claim 16. further comprising advancing said nose cone and retracting said sheath to thereby expose the intraluminal device for said delivery.
